(19) United States
(12) Patent Application Publication
(10) Pub. No.: US 2005/0228267 A1
(43) Pub. Date: Oct. 13, 2005

(54) Method and apparatus for improved breast imaging

(75) Inventors: Cherik Bulkes, Sussex, WI (US); Cynthia F. Maier, Wauwatosa, WI (US); LeRoy R. Blawat, Milwaukee, WI (US); Bernice E. Hoppel, Delafield, WI (US); John Lorbiecki, Hubertus, WI (US); Ed B. Boskamp, Menomonee Falls, WI (US); Adrian J. Knowles, Brookfield, WI (US)

Correspondence Address: JOSEPH S. HEINO, ESQ., 111 E. KILBOURN AVENUE, SUITE 1400, MILWAUKEE, WI, 53202, US

(73) Assignee: General Electric Company, Schenectady, NY (US)

(21) Appl. No.: 10/820,550

(22) Filed: Apr. 8, 2004

(51) Int. Cl. A61B 5/05

(52) U.S. Cl. 600/415

(57) ABSTRACT

A patient bed for use in performing procedures on a medical patient, comprising a lower layer with said lower layer permitting attachment to a conventional external base and an upper layer attached in a sliding relationship to said lower layer, said upper layer being angled upwardly relative to said to create a void between said upper layer, or patient support and said lower layer and having a pair of apertures in the patient support, said apertures permitting a patient's breast to fit through the aperture. The invention may also include a breast immobilization device comprising a first sliding compression plate and a second sliding compression plate, said compression plates sliding together to compress the breast of a medical patient by translating toward one another.

### BACKGROUND OF THE INVENTION

The present invention relates generally to methods and apparatus for improving the utility of magnetic resonance imaging (MRI) for breast imaging. More specifically, the invention relates to a patient support and breast immobilizing device for MRI exams and procedures. The present invention also relates to specialized RF coil elements for imaging the breast with MRI.

Magnetic resonance imaging, or MRI, is a method by which the location, size, and conformation of organs and other structures of the body may be determined. In the typical MRI system, a magnetic field is established across a body to align the spin axes of the nuclei of a particular chemical element, usually hydrogen, with the direction of the magnetic field. The aligned, spinning nuclei execute precessional motions around the aligning direction of the magnetic field. For the aligned, spinning nuclei, the frequency at which they precess around the direction of the magnetic field is a function of the particular nucleus which is involved and the magnetic field strength. The selectivity of this precessional frequency with respect to the strength of the applied magnetic field is very short and this precessional frequency is considered a resonant frequency.

In an ordinary MRI system, after the nuclei have been aligned or polarized, a burst of radio frequency energy at the resonant frequency is radiated at the target body to produce a coherent deflection of the spin alignment of the selected nuclei. When the deflecting radio energy is terminated, the deflected or disturbed spin axes are reoriented or realigned, and in this process radiate a characteristic radio frequency signal which can be detected by an external coil. The signal is then discriminated in the MRI system to establish image contrast between different types of tissues in the body. MRI systems have a variety of different excitation and discrimination modes available, such as free induction decay (“FID”), spin echo, and continuous wave, as are known in the art.

Two parameters are used to measure the response of the magnetized sample to a disturbance of its magnetic environment. One is T1, or longitudinal relaxation time, the time it takes the sample to become magnetized or polarized after being placed in an external magnetic field. The other is T2, the spin relaxation time, a measure of the time the sample holds a temporary transverse magnetization which is perpendicular to the external magnetic field. Images based on proton density can be modified by these two additional parameters to enhance differences between tissues.

There are a number of challenges to deal with in order to realize the full potential of MRI in breast imaging. For example, MRI has very high detection sensitivity for breast cancer, but is only moderately specific. That is, even when lesions are detected using MRI, it is usually not possible to obtain an accurate diagnosis either with MRI, or with other current imaging techniques, and a biopsy must be performed to remove tissue from the area of concern for further analysis. For most commercially available MRI scanners, access to the breast is extremely limited during the image acquisition, that is, while the patient is inside the magnet. To perform a biopsy of a breast lesion detected on MR images, it is therefore necessary to remove the patient from the scanner. In order to treat a lesion, what is needed is a method to reliably target a lesion detected on MR images while the patient is outside of the magnet.

This disclosure describes a device that allows imaging of a patient's breast for detection of breast cancer or other breast pathologies. It also provides localization of an area within the patient's breast that is of medical concern, and provides a method for accurately obtaining a tissue sample from this area of concern for further evaluation by use of a medical instrument such as a biopsy needle. Moreover, this device enables other therapeutic techniques such as focused ultrasound or cryotherapy to be combined effectively with the MR imaging exam. This device is compatible with existing commercially available MR scanners. It is designed to replace the conventional patient bed that is provided with the MR scanner for the duration of the breast imaging exam and related procedures.

In prior art designs, devices to facilitate biopsy of a breast lesion detected using MR were of two types: (1) portable devices that were designed to sit on top of the conventional patient bed and required the medical practitioner to reach under the patient to perform a procedure; (2) devices that were designed to replace both the patient bed and its external base which is the structure external to the magnet that supports the patient bed when it is out of the magnet.

The portable devices, for example, commercially available RF coil/biopsy modules, are all limited because they are sandwiched between the patient and the patient bed, resulting in extremely restricted access to the breast from the medial and superior approaches. In some other commercially available devices, the patient bed and external base are designed specifically for breast imaging and related procedures, precluding use of the system for other types of imaging, such as brain or spine imaging.

The optimized patient bed described here can be pulled into the magnet during imaging, and extracted from the magnet for patient set-up using the same mechanisms as the conventional patient bed. Conventionally, when the patient bed is outside of the magnet, it is physically supported on a “base”. This base is provided as an essential component of the MRI scanner, and can either be rigidly positioned in front of the magnet, or can be removeable, allowing a secondary function as a patient transport. No major modifications to conventional bases would be required for compatibility with this optimized patient bed.

### BRIEF SUMMARY OF THE INVENTION

The present invention provides a device useful for imaging a patient's breast, and, more particularly, for localizing an area of interest within the patient's breast and guiding a medical instrument during tissue-extraction procedures.

The present invention further provides for unrestricted access to a patient's breasts for many kinds of diagnostic and therapeutic procedures. The present invention provides for a patient bed that is compatible with a conventional MRI scanner such that it is not necessary to make costly modifications to the scanner for these purposes.

In general, the present invention provides a patient bed for use in a medical imaging machine comprised of: a lower layer adapted to conform to the conventional base; a patient support attached as an upper layer in a sliding relationship to the lower layer, said patient support being contoured such that the upper torso of the medical patient is supported over the lower layer, said patient support further having a pair of apertures therethrough permitting passage of the breasts of a medical patient; and a breast immobilization device comprising a first sliding compression plate and a second sliding compression plate, said compression plates sliding together to compress the breast of a medical patient by translating toward one another. The patient support could further be concave along its longitudinal access. In a preferred embodiment, the thickness of the patient support is reduced near the apertures. Additionally, in some embodiments, the lateral compression plates translate downward from the patient support as they move medially, thereby pulling the breast away from the chest wall. The compression plates may also have a surface providing a high coefficient of friction such that the breast can be gripped between the compression plates.

### BRIEF DESCRIPTION OF THE DRAWINGS

FIG. 1 is a side perspective view of an MR Imaging machine showing the magnet bore and the patient bed of the present invention.

FIG. 2 is a side perspective view showing a patient resting on the patient bed as the patient is sliding into an MR Imaging device.

FIG. 3 is a side elevational view of the patient bed of the present invention wherein the patient is has been cantilevered outward such that the breast or breasts are easily accessible for biopsy.

FIG. 4A is a front side elevational view of a patient supported by the present invention showing medial-lateral compression and cranial-caudal immobilization of the breasts.

FIG. 4B is a bottom elevational view of the right breast immobilized in FIG. 4A.

FIG. 4C is a bottom elevational view of the left breast immobilized in FIG. 4A.

### DETAILED DESCRIPTION

Referring now to the drawings in detail, wherein like numbered elements refer to like elements throughout, the present invention is generally comprised of a patient support, a device or pair of devices for immobilizing either one or both breasts during an imaging exam and associated procedures. The present invention further comprises RF coil elements for imaging both the left and right breasts using Magnetic Resonance Imaging (MRI), a method for localizing an area of concern within the breast, and a device for guiding a medical instrument during tissue-extraction procedures.

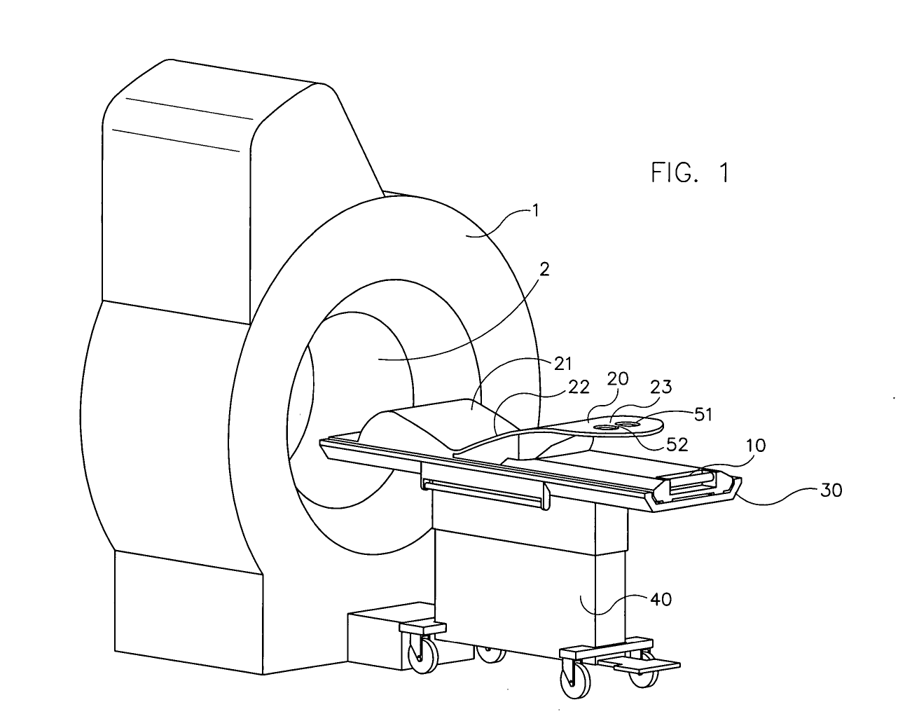

Now referring to FIG. 1, which shows a conventional MR imaging machine 1. Some MR imaging machines 1 provide for a stationary patient base that the patient is transferred to from a gurney or other conveyance. However, the particular MR imaging machine 1 shown permits the use of a mobile patient base 40.

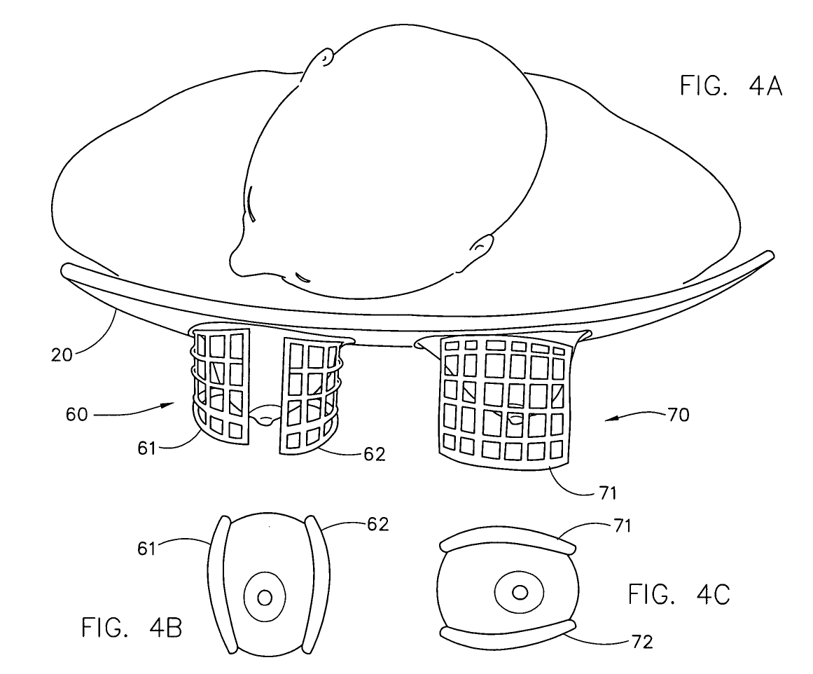

The base 40 mimics the conventional patient bed currently produced by General Electric, Inc., allowing attachment to the conventional base 40, and enabling landmarking of the patient and the ability to translate the patient bed 30 into the magnet using conventional protocols. The patient bed is generally comprised of a upper layer, or patient support, 20 and a lower layer 10 which fits into the patient bed. The improved patient support 20 is contoured so that the patient's upper body is supported above the lower layer 10, with enough space between the layers for devices that provide breast immobilization and RF coil elements. The patient's breasts can protrude through two apertures 51, 52 in the patient support 20, and are enclosed below the patient support 20 by the immobilization devices 60, 70 and RF coil elements. As shown best in FIGS. 4A-4C, the patient support 20 is concave across the area under the patient's upper body. This provides improved access to the lateral part of the breast near the chest wall relative to a flat patient support. In addition, the thickness of the patient support 20 is reduced in the lateral part of the aperture 51, 52 near the breast to further increase access to the breast tissue in this area. The patient support 20 is also curved concavely along its longitudinal access to increase patient comfort.

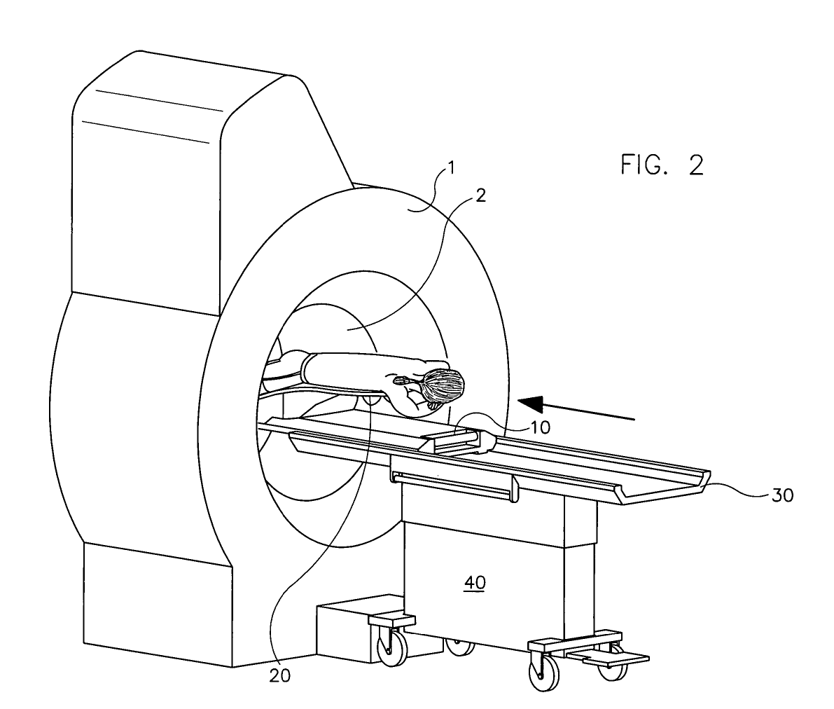

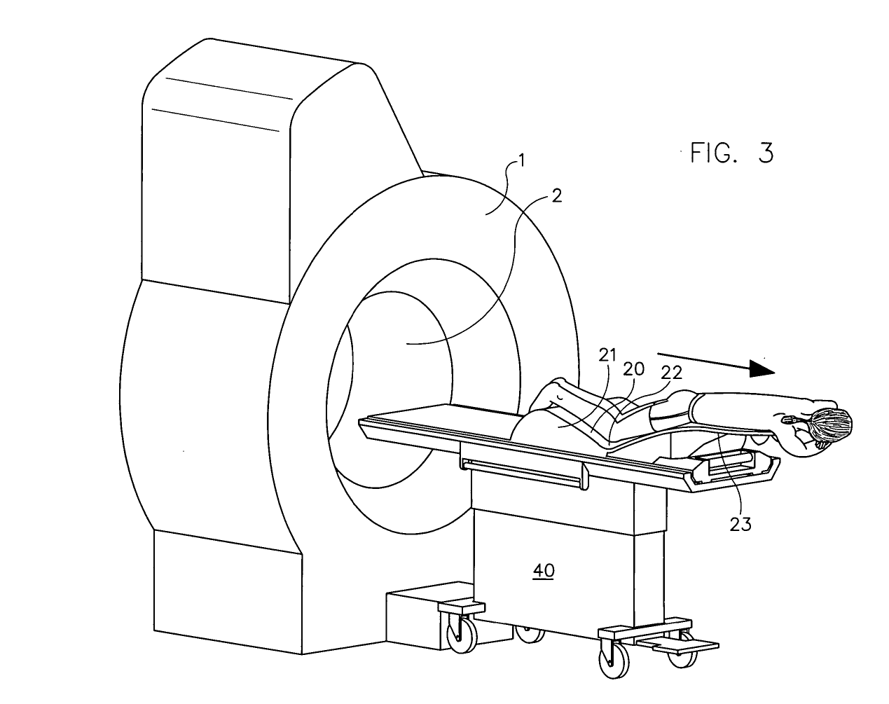

In usage, the conventional patient bed is removed and replaced before the patient is “set up” for examination. The patient bed 30 is then attached to the conventional base by replacement of the conventional patient bed with the lower layer 10. As shown in FIGS. 2 and 3, the patient bed 30 can then be put into a translation mode that enables translation of the patient support 20 relative to the patient bed 30. More specifically, the patients torso, positioned on the patient support 20 can be moved outwardly relative to the lower support such that area that houses the compression elements 60, 70 and RF coils is exposed. The desired choice of immobilization elements 60, 70 and RF coils 80 can then be attached to the bottom of the patient support 20. The patient support 20 then translates back into its “home position,” so that it is vertically aligned with the lower layer, for patient set-up and imaging.

As shown in FIG. 2, the patient support 20 can then be translated to be inserted in to the magnet bore 2 of an MR imaging machine 1 for MR breast imaging exams. In this position, the patient support 20 can be pulled into the magnet for imaging exams, so that the patient's breasts are at the isocenter of the magnet. If it is desired to add a breast biopsy procedure (or other procedure) after obtaining images, the bed 30 can be translated out of the magnet until the bed is vertically aligned with the base. The bed 30 can be put into a translation mode, so that the patient support 20 can then be further translated away from the magnet. In this position, the top half of the patient's body is cantilevered out over the distal end of the conventional base 30. This allows unrestricted access to either breast from 270 degrees, including the traditional medial, cranial and lateral approaches as well as intermediate oblique approaches.

In order to constrain the breasts in the same position during imaging and/or a biopsy (or other procedure), a device for immobilizing the breasts can be used as shown in FIG. 4A-4C. In a preferred embodiment, an easily removable immobilization device can be attached to the underside of the upper patient support 20. The immobilization device for each breast could consist of two parallel plates 61, 62 that compress the breast by translating toward each other. The top edges of these compression plates would be flush with the top surface of the upper layer of the support in order to compress the breast tissue that is close to the chest wall. The inner surfaces of the top edges of these plates could either be machined to have a textured surface, or could have a material layered on them to increase friction, thereby allowing the breast tissue to be gently gripped. The lateral plate of the pair could translate downward as it translates toward the medial plate, thereby pulling the breast tissue away from the chest wall to improve accessibility to the breast tissue for the procedure. The plates 61, 62 could either have an aperture to permit access to the breast for biopsy, or could have numerous apertures. The plates 61, 62 could consist mainly of apertures, for example in the case of grids, or a mesh. In a preferred embodiment, the compression plates 61, 62 would be curved around the breast in order to have increased area of contact with the skin and more effectively immobilize the breast. These curved plates 61, 62 could attach in two different positions to the under surface of the patient support 20, such that they compress the breast either in the medial-lateral direction 61, 62, or in the cranial-caudal 71, 72 direction.

The RF coils can be permanently attached to the bottom side of the upper layer of the patient support 20. To provide optimal access to the breast during procedures, however, the RF coils can be designed as an easily removable module that is only attached to the patient support 20 during imaging. In a preferred embodiment, the upper coil elements (i.e. the coil elements closest to the chest wall) are embedded in the lower side of the patient support. The lower coil elements are contained in a detachable module that plugs into the patient support 20 such that a continuous current path is provided through the coil via an appropriate electrical connector. Different sizes of RF coil modules can be provided so that a coil module can be selected which conforms optimally to the patient's breast size (i.e. so that the coil loading is optimized).

Typically, a biopsy will be performed only after prior imaging exams have been evaluated, and an area of concern has been identified. From evaluation of the prior imaging exams and approximating the location of the area to be evaluated (e.g. upper outer quadrant), it is possible to determine the optimal approach for the biopsy. If desired, a 3D MR imaging sequence could be used for lesion detection, allowing reformatting of the data so that it is displayed in an orientation matching the compressions that are available (or corresponding projections through the data could be displayed). With the compression plates described in our preferred embodiment, a medial-lateral 61, 62 or cranial-caudal 71, 72 compression could be used, with biopsy access to the breast from medial, lateral or cranial approaches. To perform a biopsy using a medial approach, a solid plate can be used to block the aperture corresponding to the contralateral breast. This will result in the patient being slightly rotated from the prone position.

MRI-visible reference markers can be attached to the immobilization device and/or to the patient support to provide a mechanism for translating the image reference frame into the patient support reference frame. On review of the MRI data, area(s) of concern can be identified and marked by the clinician, and this(these) location(s) can be translated into the patient support coordinate frame. These coordinates can then be used to determine the optimal approach to the area of concern using a biopsy needle (or other medical instrument), given the physical constraints placed on the procedure by the patient support 20 and associated hardware positions.

Several approaches exist and are compatible with this invention for accurately aiming the biopsy needle. For example, medical devices have been described which use, for example, infrared emitters and sensors to translate the device position and orientation into a global coordinate frame. The translation of coordinates identifying an area of concern from the patient support reference frame to the global reference frame is straightforward, but can result in inaccuracy due to multiple coordinate transformations and imprecise positioning of the patient bed in the global reference frame. In a preferred embodiment, a device to position the biopsy needle and target the area of concern could be attached to the lower side of the patient support 20. This device could be attached to the patient support 20 for the biopsy procedure after the RF coil module is removed. In one embodiment, the device could consist of a needle holder that attaches to the bottom side of the patient support on a circular rail. The needle holder could then rotate around the breast until it is at the correct angle, then the needle holder could be translated in the up/down direction until the correct position for the needle entry is obtained.

Standard procedure for MR-guided breast biopsy is to insert an MR visible device to confirm the location of the area of concern before the insertion of the biopsy needle. To perform this step, the RF coil module could be plugged into the patient support 20, or the upper coil elements could be used alone. For most patients, a strong enough MR signal would be received using the upper elements alone to provide this confirmation.

The procedure for performing a breast biopsy is described here in detail; however, it is to be understood that this device also enables other procedures such as combined ultrasound/MRI image guidance, focused ultrasound, cryotherapy, laser therapy to be combined effectively with MR imaging by providing unrestricted access to the breast.

While this invention has been described in the context of a cylindrical MRI system, it should be noted that it is also compatible with other MRI magnet designs including vertical field open systems.

A patient bed 30 for use in performing medical procedures on a medical patient, comprising; a lower layer 10, said lower layer permitting attachment to a conventional external base 40, an patient support 20 attached in a sliding relationship to said lower layer 10 and said external base 40, said patient support 20 being angled upwardly relative to said lower layer 10 and a pair of apertures 51, 52 in the patient support 20, said apertures 51, 52 permitting a patient's breast to fit through the aperture. The patient support 30 may further include a midsection 22, a leg support 21 angled upwardly from the midsection of the upper layer 22, and a torso support 23 angled upwardly from the midsection of the upper layer 22. The patient support 20 could further be concave along its longitudinal access. In a preferred embodiment, the thickness of the patient support is reduced near the apertures 51, 52. Additionally, in some embodiments, the first and second compression plates 61, 62 translate downward from the patient support 20, thereby pulling the breast away from the chest wall. The compression plates 61, 62 may also have a surface providing a high coefficient of friction such that the breast can be gripped between the compression plates 61, 62.

While various embodiments of the present invention have been described in detail, it may be that further modification and adaptations of the invention will occur to those skilled in the art. However, it is expressly understood that obvious modification and adaptations are within the spirit and scope of the present invention.

1. A patient bed for use in performing medical procedures on a medical patient, comprising:
a lower layer, said lower layer permitting attachment to a conventional external base;
an upper layer attached in a sliding relationship to said lower layer, said upper layer being angled upwardly relative to said lower layer; and
a pair of apertures in the patient support, said apertures permitting a patient's breast to fit through the aperture.

2. The patient bed of claim 1 wherein the patient support further comprises:
a midsection;
a leg support angled upwardly from the midsection of the upper layer; and
a torso support angled upwardly from the midsection of the upper layer.

3. The patient bed of claim 2 wherein said upper layer is permitted to slide relative to the lower layer such that the torso support is cantilevered outwardly from the lower layer.

4. The patient bed of claim 3 further comprising an adjustable breast immobilizer.

5. The patient bed of claim 4 wherein said breast immobilizer is comprised of:
a first compression plate; and a second compression plate for each breast, said first and second compression plates having a plurality of apertures permitting access to the breast of a medical patient.

6. The patient support apparatus of claim 5 wherein said first and second compression plates compress the breast by translating toward one another.

7. The patient support apparatus of claim 6 wherein the lateral compression plate translates downward from the patient support as it moves toward the medial plate, thereby pulling the breast tissue away from the chest wall.

8. The patient support apparatus of claim 7 wherein said first and second compression plates have a surface providing a high coefficient of friction such that the breast can be gripped between the compression plates.

9. The patient support apparatus of claim 8 wherein a left RF coil and a right RF coil are provided, said RF coils being attachable around the breasts of a medical patient.

10. A mobile patient base for use with a medical imaging machine comprising:
a lower support; said lower support sliding into the existing mobile patient base;
a patient support attached in a sliding relationship to the lower support and being contoured such that the upper torso of a medical patient is angled upwardly so as to create a void between the lower support and the patient support;
said patient support having a pair of apertures permitting the patient's breast to fit through.

11. The patient support apparatus of claim 10 further comprising an adjustable breast immobilizer positioned around the each of the apertures in the patient support, said breast immobilizer comprised of a first compression plate and a second compression plate, said first and second compression plates having a plural of apertures permitting access to the breast of a medical patient.

12. The patient support apparatus of claim 11 wherein said first and second compression plates compress the breast by translating toward one another.

13. The patient support apparatus of claim 12 wherein said first and second compression plates translate downward from the patient support, thereby pulling the breast away from the chest wall.

14. The patient support apparatus of claim 13 wherein said first and second compression plates have a surface providing a high coefficient of friction such that the breast can be gripped between the compression plates.

15. The patient support apparatus of claim 14 wherein a left RF coil and a right RF coil are provided, said RF coils being attachable around the breasts of a medical patient.

16. A patient support apparatus for use in a medical imaging machine comprising:
a patient transport;
a lower support adapted to conform to the patient transport;
a patient support attached in a sliding relationship to the lower support, said patient support being contoured such that the upper torso of a medical patient is angled upwardly such that the medical patient is supported over the lower support, said patient support further having a pair of apertures therethrough permitting passage of the breasts of a medical patient; and
a breast immobilization device comprising a first sliding compression plate and a second sliding compression plate, said compression plates sliding together to compress the breast of a medical patient by translating toward one another.

17. The patient support apparatus of claim 16 wherein said patient support is concave along its longitudinal access.

18. The patient support apparatus of claim 17 wherein the thickness of the patient support is reduced near the apertures.

19. The patient support apparatus of claim 18 wherein said first and second compression plates translate downward from the patient support, thereby pulling the breast away from the chest wall.

20. The patient support apparatus of claim 19 wherein said first and second compression plates have a surface providing a high coefficient of friction such that the breast can be gripped between the compression plates.
